(19) United States
(12) Patent Application Publication
(10) Pub. No.: US 2008/0243009 A1
(43) Pub. Date: Oct. 2, 2008

(54) METHOD OF CONTROLLING INFLATION OF A CUFF IN BLOOD PRESSURE DETERMINATION

(75) Inventors: Lawrence T. Hersh, Tampa, FL (US); Sai Kolluri, Tampa, FL (US); Bruce Friedman, Tampa, FL (US); Richard Medero, Tampa, FL (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 11/694,178

(22) Filed: Mar. 30, 2007

(51) Int. Cl. A61B 5/02

(52) U.S. Cl. 600/494; 600/495

(57) ABSTRACT

The present application discloses a method of calculating an initial inflation pressure during a blood pressure determination using an NIB system. The cuff provided with the system is inflated towards a default initial inflation pressure and a plurality of oscillometric pulses are obtained during inflation. A quick systolic pressure is estimated from a pre-defined function having a physiologically- expected shape of an oscillometric envelope fitted to oscillometric data obtained during the inflation. In an embodiment, the parameters within the function are specifically found by fitting a plurality of oscillometric pulse amplitudes along with their corresponding cuff pressures obtained during inflation to the pre-defined function. The cuff is inflated up to a calculated initial inflation pressure, which is found from the estimated quick systolic pressure. After the cuff is brought to the initial inflation pressure, deflation is begun for determining the actual systolic and diastolic pressures for output to a user.

### FIELD OF THE INVENTION

This invention generally relates to a method of controlling the cuff inflation and deflation to enhance the performance of an NIB system. More particularly, this invention relates to a method of estimating an initial inflation pressure during the cuff inflation.

### BACKGROUND OF THE INVENTION

The oscillometric method of measuring blood pressure involves applying an inflatable cuff around an extremity of a patient's body, such as a patient's upper arm. During the use of a conventional NIB monitoring system the cuff is inflated to an initial inflation pressure, which is slightly above the patient's systolic pressure. The cuff is then progressively deflated and a pressure transducer detects the cuff pressure, along with pressure fluctuations or oscillations resulting from the beat-to-beat pressure changes in the artery under the cuff. The data from the pressure transducer is used to compute the patient's systolic pressure, mean arterial pressure (MAP) and diastolic pressure. As can be understood, the selection of the initial inflation pressure is an important factor in determining the amount of time required by the NIB system to measure cuff pressure and to detect cuff oscillations for the estimation of blood pressure.

A key requirement in determining the blood pressure using an NIB monitoring system is that the cuff needs to be inflated above the systolic pressure so that a good representation of the oscillation amplitude pattern can be measured. If a recent blood pressure has already been measured, the systolic information from that previous determination can be used to determine the initial inflation pressure for the present determination. However, this technique cannot be used if the last determination is not recent, or the patient has been changed, or the instrument has just been powered on. In other words, the determination must be done with no a priori knowledge of an estimate of the blood pressure.

This means that the initial inflation pressure may not be optimal for the particular circumstances being measured. In order to handle this, the system must pump up to a high pressure to try to guarantee that it is above systolic. Alternatively, the system must upon observing the oscillation pattern during the deflation decide that there is not enough information at the high cuff pressure end of the measured oscillometric data to reasonably estimate systolic; this requires further pumping and searching. These scenarios waste time and cause discomfort for the patient.

Thus if the initial inflation pressure is selected well above the systolic blood pressure for the patient, the NIB system over inflates the blood pressure cuff, resulting in patient discomfort and extended measurement time. Alternatively, if the initial inflation pressure is selected below the systolic blood pressure for the patient, the blood pressure cuff must re-inflate to obtain an accurate reading. Therefore, it is desirable to have some knowledge of the patient's blood pressure in order to control the cuff inflation and deflation to enhance the performance of an NIB system.

As can be understood, the selection of the initial inflation pressure determines the amount of time required before the NIB system begins to deflate the cuff pressure for the purpose of measuring cuff pressure along with detecting cuff pressure oscillations to estimate the patient's blood pressure. Thus there exists a need to specify the initial inflation pressure during cuff inflation, for controlling the inflation of the cuff.

### SUMMARY OF THE INVENTION

The above-mentioned shortcomings, disadvantages and problems are addressed herein which will be understood by reading and understanding the following specification.

The present invention discloses a method of calculating an initial cuff inflation target pressure while monitoring blood pressure. The method comprises the steps of: (a) inflating a blood pressure cuff; (b) monitoring for the presence of oscillometric pulses during the inflation of the blood pressure cuff; (c) estimating a systolic blood pressure for a patient based on the oscillometric pulses detected during the inflation of the blood pressure cuff; and (d) defining the initial inflation pressure as a predetermined pressure above the estimated systolic pressure; wherein the systolic blood pressure is estimated by a curve fitting using a function having a physiologically-expected shape of an oscillometric envelope, the curve fitting being done by using a plurality of oscillometric pulses along with corresponding cuff pressures obtained during early part of inflation.

In another embodiment, a method of monitoring blood pressure in a patient is described. The method comprises the steps of: (a) providing a non-invasive blood pressure (NIB) monitor having a selectively inflatable and deflatable blood pressure cuff and at least one pressure transducer for detecting oscillometric pulses; (b) inflating the blood pressure cuff; (c) monitoring for the presence of oscillometric pulses from the pressure transducer during the inflation of the blood pressure cuff; (d) estimating the MAP and diastolic pressures for the patient by a curve fitting using a pre-defined function having a physiologically-expected shape of an oscillometric envelope, the function parameters being found by fitting a plurality of oscillometric pulses obtained during inflation along with the corresponding cuff pressures to the fitted curve; and (e) terminating the inflation of the blood pressure cuff at an initial inflation pressure above the estimated systolic blood pressure, where the systolic pressure is estimated from a calibrated cuff pressure oscillation or from a mathematical formula that relates systolic to diastolic and MAP.

In yet another embodiment, a method of controlling inflation of a cuff during monitoring blood pressure of a patient using a NIB system is disclosed. The method comprises the steps of: (a) providing the patient with a selectively inflatable and deflatable non-invasive blood pressure cuff arranged to be worn about a limb of the patient and operatively connected to a non-invasive blood pressure monitor; (b) estimating a quick systolic blood pressure for the patient from oscillometric information based on only part of the inflation pressure range, the quick systolic being found by curve fitting oscillation amplitude along with corresponding cuff pressure data to a pre-defined function having a physiologically expected shape of an oscillometric envelope; and (c) controlling inflation of the blood pressure cuff based on a calculated initial inflation pressure, the initial inflation pressure being calculated from the estimated systolic pressure, during inflation of the blood pressure cuff.

### BRIEF DESCRIPTION OF THE DRAWINGS

A clear conception of the advantages and features constituting inventive arrangements, and of various construction and operational aspects of typical mechanisms provided by such arrangements, are readily apparent by referring to the following illustrative, exemplary, representative, and non-limiting figures, which form an integral part of this specification, in which like numerals generally designate the same elements in the several views, and in which:

FIG. 1 illustrates a non-invasive blood pressure (NIB) monitoring system capable of implementing a method of estimating an initial inflation pressure described in an embodiment of the invention;

FIG. 2 is a graph depicting the signals generated during a typical blood pressure determination which includes some over-inflation of the blood pressure cuff relative to the systolic pressure;

FIG. 3 is a graph depicting an oscillometric envelope seen while monitoring the blood pressure and showing the time periods for inflation and deflation;

FIG. 4 is a graph depicting in detail, a part of the oscillometric envelope seen during inflation of a cuff and showing an example of where the oscillations arise for calculating the initial inflation pressure;

FIG. 5 is a graph depicting the estimation of systolic pressure from an osillometric pulse calibrated during the inflation period of a blood pressure determination as disclosed in an embodiment of the invention;

FIG. 6A is a flowchart illustrating the method of calculating an initial inflation pressure, while monitoring the blood pressure of a patient as described in an embodiment of the invention;

FIG. 6B is a flowchart illustrating the method of calculating an initial inflation pressure, while monitoring the blood pressure of a patient as described in another embodiment of the invention;

FIG. 7 is a flowchart illustrating the method of determining the blood pressure of a patient as described in an embodiment of the invention; and

FIG. 8 is a flow chart illustrating the course of actions in estimating the initial inflation pressure during cuff inflation as in an embodiment of the invention.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

In various embodiments, a method of estimating the patient's blood pressure to control the cuff inflation and deflation to enhance the performance of a NIB system is disclosed. A systolic pressure is estimated from a minimum number of oscillations obtained during the inflation of the cuff. These oscillations arise during the early part of the inflation period and cover only part of the oscillometric pressure range usually associated with estimating blood pressure. From the early inflation systolic pressure estimate, an initial inflation pressure is obtained. The terms initial inflation pressure, initial target pressure, initial pump up pressure, etc. are synonymous and indicate the pressure up to which a cuff needs to be inflated for obtaining a good oscillometric pattern for determining the blood pressure.

In an embodiment, the invention provides a method of preventing patient discomfort while monitoring the blood pressure due to over inflation of the cuff, by controlling the inflation. The invention provides a quick estimation of the systolic pressure during the inflation by fitting a pre-defined function curve to oscillation amplitudes along with the corresponding cuff pressures obtained during the early part of inflation. The initial inflation pressure is selected slightly above the systolic pressure, wherein the systolic pressure is estimated during the inflation from information derived from the fitted curve.

In an embodiment, the invention provides a method of quick estimation of the blood pressure on inflation for use in calculating the initial inflation target. Normally, the blood pressure output by the NIB monitor is determined during the deflation period by deflating the cuff pressure either continuously or incrementally in a series of small steps. The oscillometric envelope built during the deflation period is carefully constructed so that the published output is as accurate as possible. To find an accurate blood pressure, the cuff pressures that must covered during the determination should have a range that includes the actual intra-arterial systolic and diastolic pressures. However, a quicker, but rougher estimate of blood pressure can be made if fewer oscillations are used and the requirement to cover the full range usually needed is relaxed. This can be done during the short inflation period. The goal is only to find an optimal initial inflation pressure, not to change the usual technique for estimating oscillometric blood pressure. Since the method described in the invention calculates an optimal initial inflation pressure, during inflation, it avoids the discomfort and the time wasted due to over inflation. The systolic blood pressure is estimated from a curve with a pre-defined function, fitted to a minimum number of oscillations covering only a part of the oscillometric pressure range obtained during inflation. The initial inflation pressure is estimated during the inflation as opposed to using a pre-defined level or basing it on a previous blood pressure estimate as has been done in the past.

The invention provides a technique for obtaining the initial inflation pressure, independent of any default initial inflation pressure or any prior knowledge of an estimate of blood pressure from any previous measurements so that determination time is reduced and patient comfort is increased.

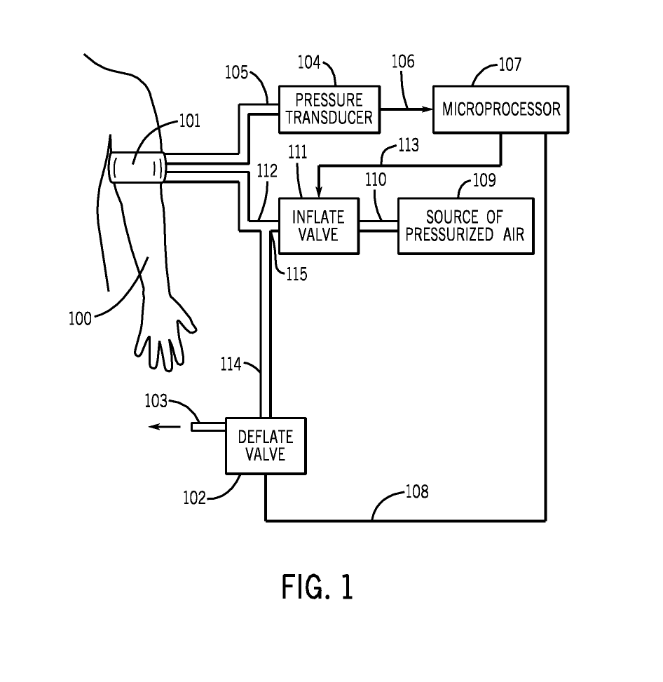

FIG. 1 illustrates a non-invasive blood pressure (NIB) monitoring system capable of implementing a method of estimating an initial inflation pressure described in an embodiment of the invention. The NIB monitoring system 100 includes a blood pressure cuff 101 placed on the arm of a patient. The blood pressure cuff 101, herein after referred to as the cuff or pressure cuff can be inflated and deflated for occluding the brachial artery of the patient when in the fully inflated condition. As the blood pressure. cuff 101 is deflated using the deflate valve 102 through a duct 114, having exhaust 103, the arterial occlusion is gradually relieved. The deflation of the blood pressure cuff 101 by the deflate valve 102 is controlled by a microprocessor 107 through the control line 108.

A pressure transducer 104 is coupled by duct 105 to the blood pressure cuff 101 for sensing the pressure within the cuff 101. In accordance with general oscillometric techniques, the transducer 104 is used to sense pressure oscillations in the cuff 101 that are generated by pressure changes in the brachial artery under the cuff. The electrical oscillations from the pressure transducer 104 are obtained by the microprocessor 107, using an analog-to digital converter, through connection line 106.

A source of pressurized air 109, such as an air compressor or compressed gas cylinder, is connected directly or indirectly to the inflation cuff 101. If the source of pressurized air is supplied by a compressed gas cylinder, an inflate valve 111 is positioned between the source 109 and the duct 112. The operation of the inflate valve 111 is controlled by the microprocessor 107 through the control line 113. Thus, the inflation and deflation of the blood pressure cuff 101 is controlled by the microprocessor 107 through the deflate valve 102 and the inflate valve 111, respectively. However if the source of pressurized air 109 is an air compressor, the air compressor may be coupled directly to a duct 112, which directly connects to the cuff 101 for inflation.

For monitoring, the blood pressure the cuff 101, wound around the patient's upper arm, is inflated from approximately zero pressure to an initial inflation pressure. Once the cuff is inflated the microprocessor 107 receives oscillations from the pressure transducer 104 and the amplitude of the oscillations along with the corresponding cuff pressures are stored in a memory (not shown) in the microprocessor 107. Once the cuff 101 is inflated up to the initial inflation pressure, the deflate valve 102 is actuated and the cuff pressure is released. During deflation the microprocessor 107 detects the oscillations and eventually estimates the systolic pressure, mean arterial pressure (MAP), and diastolic pressure.

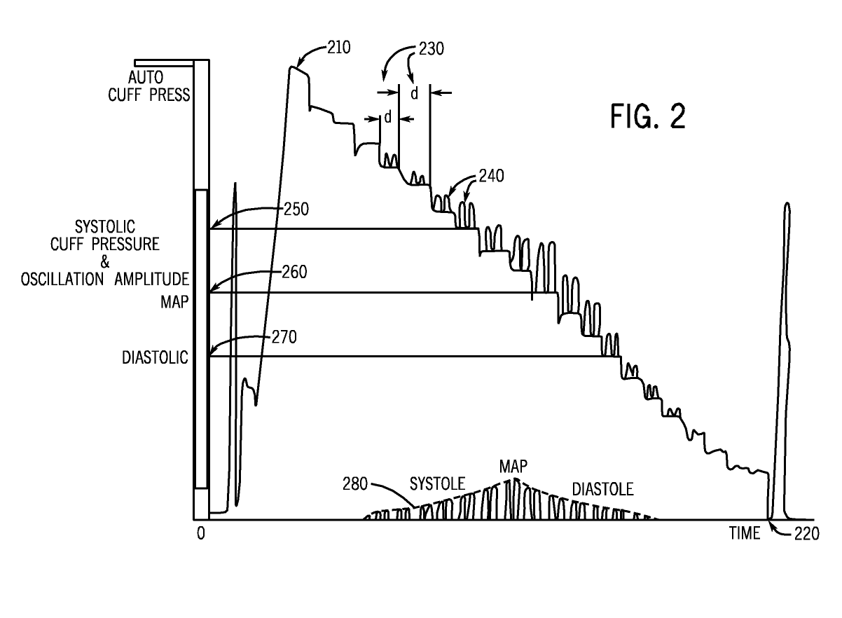

FIG. 2 is a graph depicting the signals generated during a typical blood pressure determination that includes some over inflation of the blood pressure cuff relative to the systolic pressure. For monitoring blood pressure using an NIB monitoring system the blood pressure cuff is initially placed on the patient, typically around the subject's upper arm over the brachial artery. At the inception of the measuring cycle, the blood pressure cuff is inflated from approximately zero pressure to an initial inflation pressure 210. After the blood pressure cuff has been inflated to the initial inflation pressure 210, which is calculated by a method as disclosed in an embodiment of the invention, the deflate valve is actuated by the microprocessor to deflate the cuff to a final pressure 220. The deflation may be done by releasing the cuff pressure in a series of constant pressure steps 230. Although various values for each pressure step 230 can be utilized, in one embodiment of the invention, each pressure step 230 is about 8 mm Hg per step.

After each pressure step 230, the NIB monitoring system detects and records one or more pressure oscillations 240 for the current cuff pressure level. The pressure transducer measures the internal cuff pressure and provides an analog signal characterizing the blood pressure oscillations. The peak values of the oscillations are determined within the microprocessor.

Although typical cuff pressure control of the NIB monitoring system is shown in FIG. 2 as including distinct pressure steps 230 from the initial inflation pressure 210 to a final pressure 220, the NIB monitoring system could also operate with a continuous, smooth, or linear pressure profile from the initial inflation pressure 210 to the final pressure 220. As the cuff pressure decreases from the initial inflation pressure, the NIB monitoring system detects pressure oscillations 240 and records the pressure oscillations for the current cuff pressure. Using this information, the microprocessor within the NIB monitoring system can then estimate systolic pressure 250, mean arterial pressure (MAP) 260, and diastolic pressure 270.

As the measurement cycles progress, the peak amplitude of the oscillations generally become monotonically larger to a maximum and then become monotonically smaller as the cuff pressure continues toward full deflation, as illustrated by a bell-shaped graph 280. The peak amplitude of the cuff pressure oscillations, and the corresponding occluding-cuff pressure values, are retained in the microprocessor memory. The details of the oscillations are used by the microprocessor to calculate the systolic pressure 250, the mean arterial pressure (MAP) 260 and the diastolic pressure 270 in a known manner.

As can be understood in the graph of FIG. 2, the initial inflation pressure 210 for the blood pressure cuff must exceed the systolic pressure 250 of the patient for the system and method of the NIB monitoring to function effectively. In past embodiments of the NIB monitoring systems, the initial inflation pressure 210 is either based upon the systolic pressure 250 determined during the last measurement cycle or is set at a constant value for each patient. The systolic pressure 250 from the last measurement cycle is typically increased by a set value or percentage to determine the initial inflation pressure 210 for the next measurement cycle. Since the last blood pressure cuff measurement may have been taken at a significant time period before the current measurement, the initial inflation pressure based upon the last measurement may be incorrect due to changing conditions relative to the patient. Further, if a standard value is used for the patient, the initial inflation pressure 210 may be much too high or even to low, depending upon the patient. In the case of the initial (or only) blood pressure measurement for the patient, there is no prior measurement from which to derive the initial inflation pressure 210. In such case, the prior art system relies upon a standard value, which is the same for every patient.

In the graph of FIG. 2, the initial inflation pressure 210 is selected significantly higher than the systolic pressure 250. In this operating example, the pressure within the blood pressure cuff must be decreased a significant number of pressure steps 230 before the cuff pressure reaches the systolic pressure 250. The over inflation of the blood pressure cuff results in the patient experiencing discomfort due to unnecessarily high cuff pressures and prolonged occlusion of the brachial artery. Further, the over inflation of the blood pressure cuff increases the overall time required to take a blood pressure reading from the patient due to the numerous pressure steps 230 required before the cuff pressure reaches the systolic pressure 250.

In addition to the over inflation, the initial inflation pressure 210 can be incorrectly selected to be below the systolic pressure 250. If the initial inflation pressure 210 is below the systolic pressure 260, the NIB monitoring system will not obtain the required oscillometric pressure measurements needed to accurately calculate the systolic pressure 260. In this situation, the NIB monitoring system must re-inflate the blood pressure cuff to an inflation pressure that is greater than the systolic pressure 260. In such a situation, the patient again experiences prolonged blood pressure determination time and increases discomfort.

Although the method of calculating the initial inflation pressure based upon earlier blood pressure determinations is generally effective, the initial inflation pressure 210 may be in error if the patient's blood pressure has changed significantly in the time between the current NIB measurement and the previous NIB determination. In some cases, the amount of time between blood pressure measurements may be 15 minutes to an hour. If the patient's blood pressure has changed significantly in that time period, the standard inflation adjustment may be incorrect and result in either over inflation or under inflation, thereby prolonging the blood pressure determination cycle.

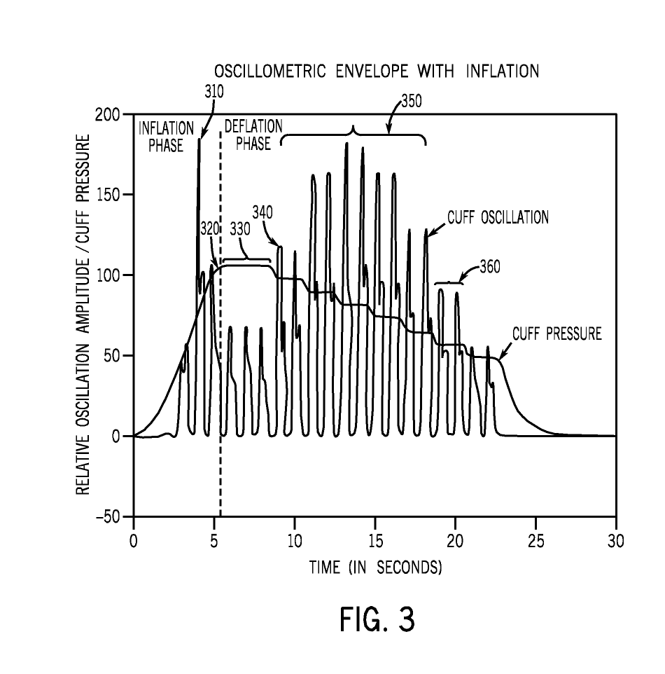

FIG. 3 is a graph depicting an oscillometric envelope seen while making a blood pressure determination and showing the time periods for inflation and deflation. During monitoring the blood pressure, a blood pressure cuff is typically wound around the subject's upper arm over the brachial artery. During initial inflation of the blood pressure cuff, the pressure transducer in the NIB system generates oscillations that are received by the microprocessor. Typically, the blood pressure cuff is inflated rapidly from approximately zero pressure to an initial inflation pressure, which is slightly above the systolic pressure. When the NIB monitor begins the process of inflating the pressure cuff, a pressure transducer is used to detect the oscillations. Conventional digital filter techniques may be used to yield oscillometric pulses corresponding to each heartbeat. Upon receiving the filtered signal, the microprocessor is able to detect oscillometric pulses present during the inflation of the blood pressure cuff.

During the inflation phase of the blood pressure determination a plurality of oscillometric pulses are obtained and the amplitude of the oscillometric pulses increases as the cuff pressure increases. As the measurement cycles progress, the cuff pressure is gradually increased and the peak-to-peak amplitude of the oscillometric pulses generally become monotonically larger to a maximum, shown as 310. The cuff pressure at this maximum value approximates MAP. The cuff is further inflated to a pressure that fully occludes the brachial artery, i.e., prevents blood from flowing through the brachial artery at any point in the heart cycle. The amplitude of the oscillation starts decreasing, shown as 320 once it reaches the maximum and at this point during the inflation phase the curve fitting can be done to estimate the systolic pressure. With the pressure cuff inflated beyond the systolic pressure, the artery is completely occluded and no blood can flow through it. Typically the cuff pressure is increased to an initial inflation pressure, which is slightly above the systolic pressure so that a complete set of oscillometric pluses can be obtained during the deflation phase of the determination for accurately determining the blood pressure.

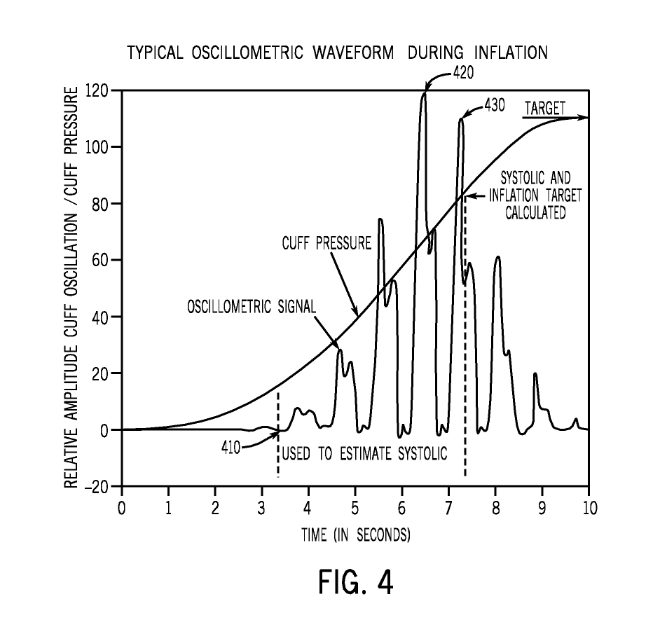

In an embodiment, the estimation of the systolic pressure is done during the decrease of the amplitude of the oscillations from 310 to 320 during the inflation. The technique of curve fitting is explained in reference to FIG. 4. Once the cuff is inflated to the calculated or default initial inflation pressure, the deflate valve of the NIB system is actuated by the microprocessor to deflate the cuff in a series of constant pressure steps. The blood pressure is determined at the end of the deflation period from the oscillations obtained during the deflation period. Typically the cuff pressure is deflated slowly by steps and during the initial phase of deflation, i.e at 330, the amplitude of the oscillations received is minimal. At point 340, the oscillation size has grown to the level that will later be determined as the systolic point of the oscillometric envelope to determine the systolic pressure. The amplitude of the oscillations at this point is a fixed fraction (ratio) of the maximum oscillation amplitude found at MAP. The amplitude of the oscillations keeps on increasing as the cuff pressure is released due to the increased flow of blood through the artery and reaches a maximum and then starts decreasing shown as the period 350. As the cuff pressure is released further and at a point 360 an oscillation size is reached which will later be determined as the diastolic point of the oscillometric envelope to determine the diastolic pressure. Once the diastolic pressure level is reached within the cuff, the amplitude of the oscillations has a magnitude that is a fixed fraction (ratio) of the size of the oscillations found at MAP.

In an embodiment, from a pre-defined function such as a Gaussian, having a physiologically-expected shape of an oscillometric envelope, by curve fitting a plurality of the oscillometric pulse amplitudes and the corresponding cuff pressures from the initial portion of an inflation period, the systolic pressure can be estimated. For the purposes of this invention, generally, the systolic pressure is estimated at that point during inflation period at which the amplitude of the oscillations first starts decreasing from the maximum during the inflation i.e. during the transition of oscillations from 310 to 320. From the estimated systolic pressure the initial inflation pressure is calculated and the microprocessor uses this value to control the termination of inflation of the cuff at 320.

FIG. 4 is a graph depicting, in detail, the oscillometric envelope seen during inflation of a cuff and showing an example of where the oscillations arise for calculating the initial inflation pressure. The systolic pressure is estimated for use in calculating the initial inflation pressure, which is needed to obtain the complete oscillation pattern later during deflation. The determination of the blood pressure for publication and output to the user is done at the end of the deflation period. During the inflation, the cuff pressure is increased from approximately zero to an initial inflation pressure. During inflation, the cuff pressure is slowly increased and at point 410, the microprocessor starts receiving oscillations from the pressure transducer. As the cuff pressure increases the amplitude of the oscillations keeps on increasing and at point 420, the amplitude of the oscillations become a maximum. The cuff pressure at this point estimates the MAP. Once the oscillation amplitude reaches the maximum, the amplitude of the oscillations start decreasing as indicated at 430. The oscillations obtained during a period of inflation, i.e. at 410 to the point at which amplitude of the oscillations start decreasing i.e. at 430, are taken for estimating the systolic pressure. The amplitudes of these oscillations, as well as the applied cuff pressure, are stored together as the system automatically changes the cuff pressure over the range of interest. These peak-to-peak amplitudes of the oscillations define an oscillometric envelope and are evaluated to find the maximum value and its related cuff pressure, which is approximately equal to the MAP. The cuff pressure below the MAP value that produces a peak-to-peak complex amplitude having a certain fixed relationship to the maximum value, is designated as the diastolic pressure. Likewise, the equivalent cuff pressure above the MAP value that results in oscillations having an amplitude with a certain fixed relationship to that maximum value, is designated as the systolic pressure. The relationships of systolic and diastolic pressures, respectively, to the maximum value, are empirically derived ratios that assume varying levels depending on the preferences of those of ordinary skill in the art. The systolic pressure estimated using the inflation period, is done by visiting cuff pressures below a value slightly greater than the MAP; this means that this quick inflation systolic estimate is predictive and can be used in calculating the initial inflation pressure.

In an embodiment, the systolic pressure can be estimated by first curve fitting a pre-defined function having a physiologically-expected shape of an oscillometric envelope to a plurality of oscillometric pulse amplitudes obtained during inflation along with their corresponding cuff pressures, the parameters within the function being found by the curve fitting. Essentially, the measured oscillometric data is used to adjusted parameters within the pre-defined function until an optimal fit is achieved. Knowing the optimal values for the parameters and the pre-defined function itself, a fitted curve can be completely defined. The fitted curve can then be easily used to get an approximation of the mean arterial pressure (MAP) data point, which is approximately at the maximum value of the fitted curve. From this maximum value data point, the systolic and diastolic pressures may be computed as those pressures which have oscillation amplitudes that are fixed percentages of the maximum oscillation value occurring at MAP. In this manner, the systolic data point and the diastolic data point along the fitted curve may each be computed and therefore their respective pressures may be determined. Since the curve fit is done immediately upon completion of the early inflation period 430, well before achieving a cuff pressure in the vicinity of the actual systolic pressure, it can be used to help calculate the target inflation. For example, the systolic pressure can be estimated by using the MAP and the diastolic pressure from the curve fit along with the well known mathematical relationship that often exists among systolic, MAP, and diastolic, such as the systolic estimate equals the diastolic estimate plus three times the MAP-diastolic difference. Once the systolic pressure has been estimated it can be used to help determine the initial inflation pressure.

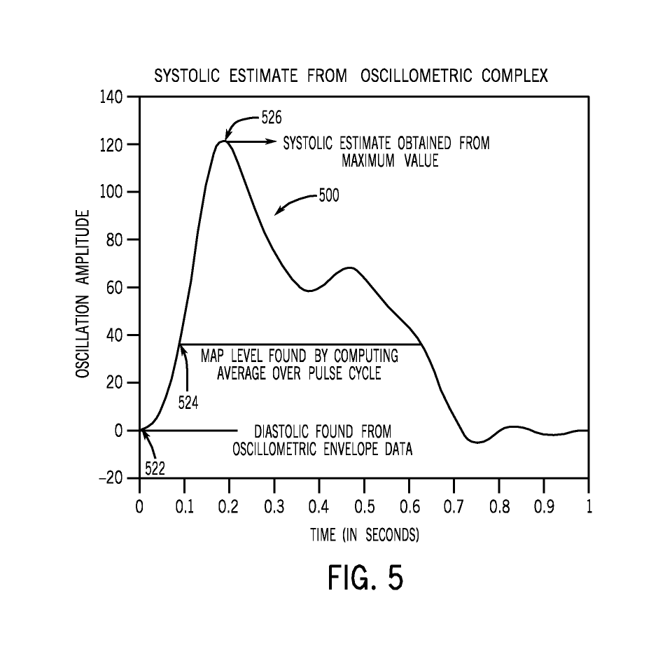

FIG. 5 is a graph depicting the estimation of systolic pressure from an oscillometric pulse calibrated during the inflation period of a blood pressure determination as disclosed in an embodiment of the invention. An oscillometric pulse 520 is selected for estimating the systolic pressure by this alternative technique. The systolic pressure may be estimated by first fitting a pre-defined function using the plurality of oscillations and corresponding cuff pressures obtained during the early part of inflation. Next, the MAP and diastolic pressure can be estimated from the fitted curve. These pressures can be estimated using the techniques specified earlier. The MAP level 524 on the oscillometric waveform may be identified by computing a time average value of the oscillometric pulse cycle. The diastolic level 522 on the oscillometric waveform may be identified as the minimum value of the oscillometric cycle. Once the MAP 524 and the diastolic 522 levels are found, the estimated MAP and diastolic pressure from the curve fitting can be associated with those levels and then the systolic pressure can be found from the systolic level 526. Again, once the systolic pressure has been estimated it can be used to help determine the initial inflation pressure. Typically, the oscillation with maximum amplitude is selected for calibration. The systolic level 526 is found as the maximum that occurs in the oscillometric waveform during the heart cycle.

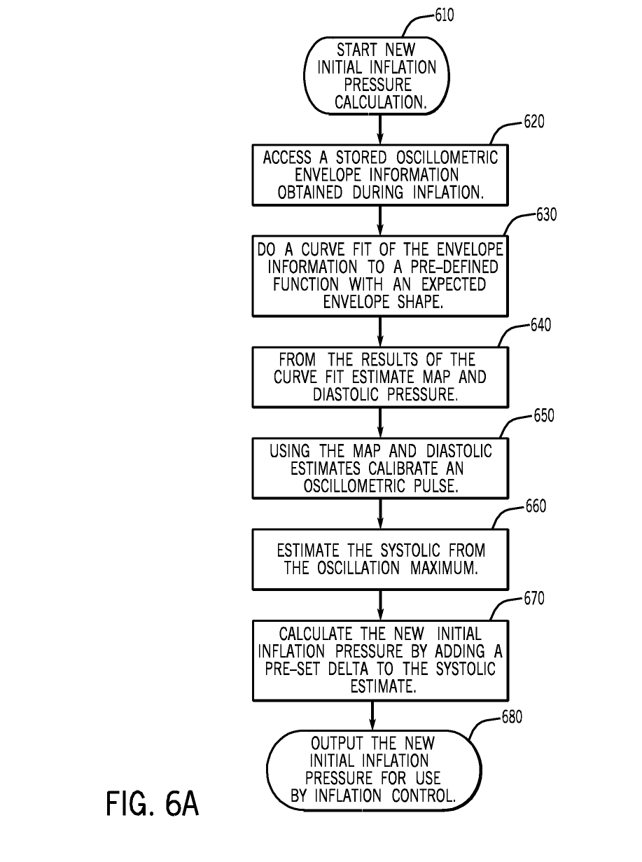

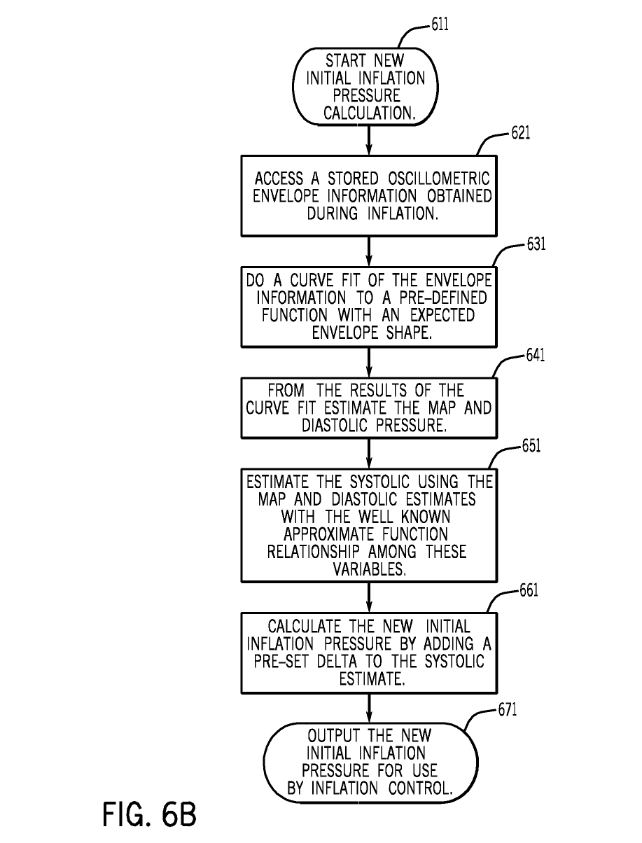

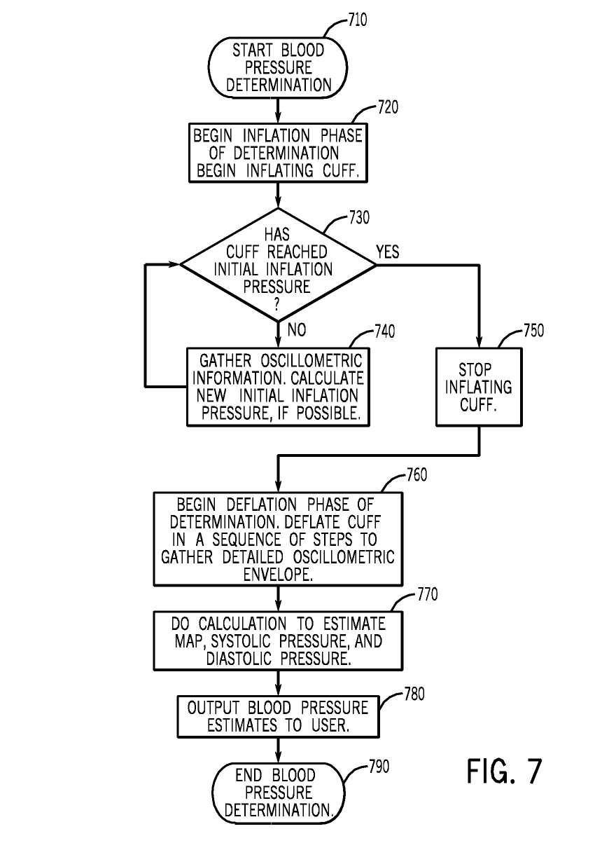

FIG. 6A is a flowchart illustrating the detailed method of calculating an initial inflation pressure for estimating the blood pressure of a patient using an oscillometric technique as is described in an embodiment of the invention. At step 610, the process of calculating a new initial inflation pressure is started. The algorithm represented by FIG. 6A is executed when it may be possible to calculate a new initial inflation pressure, i.e. at step 430 of FIG. 4. Alternatively, the algorithm represented by FIG. 6A fits into the blood pressure determination at step 740 of FIG. 7. The entirety of FIG. 6A is a more detailed representation of step 740 of FIG. 7, which will be explained below. At step 620, oscillometric information obtained during inflation is accessed. This step includes obtaining from the microprocessor memory information about a plurality of oscillations that have occurred during inflation. Using filters, the NIB monitor detected, measured, and stored oscillometric pulse amplitude information derived from the cuff pressure waveform during the inflation period. The step further includes defining an oscillometric envelope using the plurality of oscillometric pulse amplitudes along with their corresponding cuff pressures obtained during the early part of inflation that were also stored in the memory of a microprocessor. At step 630, a curve fitting of the oscillometric envelope information to a predefined function having a physiologically-expected shape of an oscillometric envelope is done. For example, a Gaussian function may be used as the pre-defined function. While the cuff is inflating the amplitude of the oscillations will increase and reach a maximum and then start decreasing. At this point, there is enough oscillometric information so that the curve fit can be done which can then give or help to give a quick estimate of the systolic pressure. The systolic pressure is estimated preferably before the cuff pressure reaches a default initial inflation pressure so that it is useful in helping to calculate a better inflation target pressure. At step 640, from the curve fit the MAP and diastolic pressure are estimated. At step 650, the systolic is estimated from the estimated MAP and diastolic. This step includes selecting an oscillometric pulse obtained during inflation for calibrating the same to estimate the systolic pressure. The oscillometric pulse is calibrated with the MAP and diastolic estimated from the curve fit. Typically the oscillometric pulse obtained with maximum amplitude is selected for calibration. At step 660, the systolic pressure is estimated from the maximum of the calibrated oscillometric pulse waveform. At step 670, based on the estimate of systolic, a new initial inflation pressure is calculated. This is achieved by adding a pre-set delta to the estimated systolic pressure. At step 680, the new initial inflation target pressure is used in controlling the inflation. The new initial inflation pressure is used only if the new initial inflation pressure is estimated before the cuff pressure achieves the default initial inflation pressure.

FIG. 6B is a flowchart illustrating an alternative embodiment of calculating an initial inflation pressure when monitoring blood pressure of a patient using the oscillometric technique. Steps 611 through 641 are the same as steps 610 through 640 of FIG. 6A. However, at step 651, the systolic pressure is estimated by a different means. Specifically, the quick systolic pressure estimate is calculated by using the MAP and diastolic obtained from the curve fit along with a well known approximate mathematical formula relationship as described earlier in this specification. Step 661 and 671 are the same as steps 670 and 680 of FIG. 6A. In this way, FIG. 6B illustrates how different means of quickly estimating systolic pressure during inflation fit into the overall blood pressure algorithm.

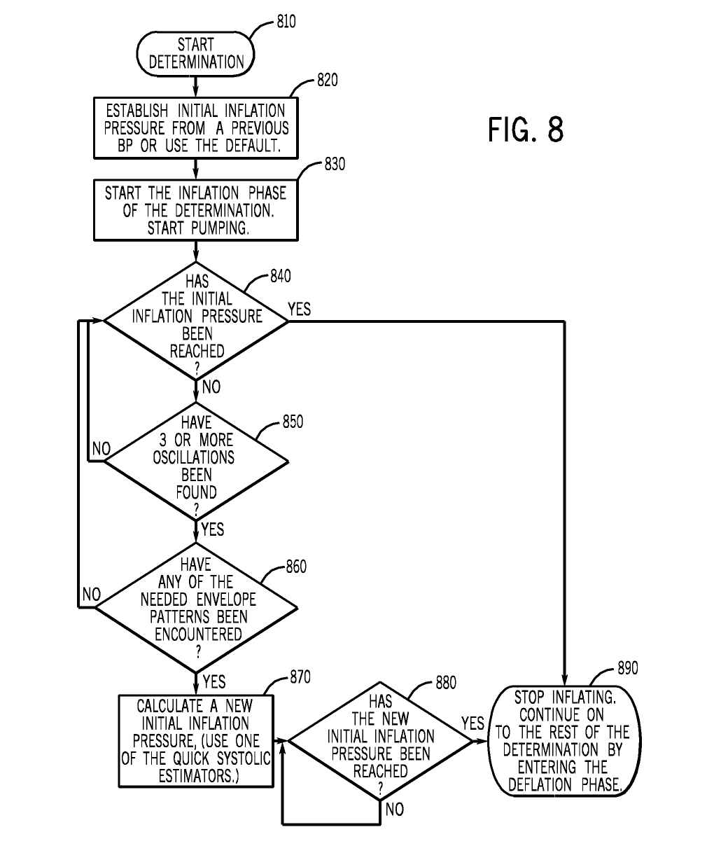

FIG. 7 is a flowchart illustrating the method of measuring the blood pressure of a patient as described in an embodiment of the invention. In FIG. 7, steps 720 to 750 show the inflation phase of the blood pressure determination, and steps 760 to 780 show the deflation phase of the determination. At step 710, blood pressure determination is started. At step 720, cuff inflation is started from a zero pressure and a default initial inflation pressure is set. The cuff is inflated at a rate so that a sufficient number of oscillations are obtained for estimating the necessary blood pressure values during inflation. At step 730, the system checks whether the cuff pressure has reached the initial inflation pressure set by the system at the beginning of the blood pressure determination. If the cuff pressure has already reached the default initial inflation pressure, cuff inflation is terminated as shown at step 750. At step 740, if the cuff has not yet reached the default initial inflation pressure, then, if possible, a new initial inflation pressure is calculated from the oscillometric information. The new initial inflation pressure is calculated by any one of the methods described in FIGS. 6A and 6B. The actions that need to be taken while in inflation phase of the determination are shown with more detail in FIG. 8. If a new initial inflation pressure is calculated, it will then be used for pumping the cuff to the highest needed level rather than the default initial inflation pressure. Once the cuff pressure reaches either the newly calculated initial inflation pressure or the default initial inflation pressure, the inflation of the cuff is terminated as indicated by step 750. If a new initial inflation pressure has been calculated, whether it is greater than or less than the first initial inflation pressure, it takes precedence in controlling the termination of the pumping. However, it is possible to update the new initial inflation pressure if the inflation period is long enough. As more oscillations are gathered during the inflation period better new initial inflation pressures can be calculated. At step 760, the cuff deflation is begun. The cuff is deflated from the initial inflation pressure to a much lower level. A plurality of oscillometric pluses is monitored during the deflation to get an oscillometric envelope corresponding to the deflation. At step 770, from the oscillometric pluses the systolic, MAP and diastolic pressure are calculated for publication and output to the user. At step 780, the output estimate of the blood pressure is provided to the user and at step 790, the blood pressure determination is concluded.

In an embodiment, the quick systolic is estimated by a curve fit using a pre-defined function. The parameters within the pre-defined function are found by the curve fitting algorithm which uses a plurality of oscillometric pulse amplitudes as cuff pressure changes over the early inflation period. Any well known curve fitting algorithm can be used. For example, the Marquardt-Levenberg algorithm could be easily implemented in this invention. In order to do the calculation for the quick estimate of systolic, the oscillometric envelope defined by the oscillation amplitudes versus cuff pressure data must have a reasonable bell shape. This may be ensured by requiring that there are three or more oscillations, with one oscillation having a clear amplitude maximum, and the diastolic side oscillation must have an amplitude less than one half the maximum, and the systolic side oscillation must have an amplitude less than 0.9 the maximum. Alternatively, there must be more than three oscillations with at least one oscillation clearly on the systolic side of the envelope. If the measured oscillometric data meets these requirements a curve fit can be undertaken for estimating the various needed blood pressure estimates. The MAP and diastolic are estimated from the fitted curve. The cuff pressure at which the amplitude of the oscillations become maximum is the MAP estimate and the cuff pressure at which the amplitude of the oscillations is at 60% of the MAP oscillation size on the lower pressure side of the envelope is the diastolic estimate. The 60% diastolic point can be easily obtained once the fitted curve is completely defined and is meant only as an illustrative example; some other percentage or ratio may be preferred by those skilled in the art.

In an embodiment, as described earlier, the quick systolic pressure can be estimated using a mathematical formula known as the “one-third rule”. Specifically, systolic is estimated by the diastolic plus three times the MAPdiastolic difference. This rule normally is used to estimate MAP as the diastolic plus one-third the pulse pressure, but it can be algebraically manipulated, for the purposes of this invention, to estimate systolic from the diastolic and the MAP. Also, as described earlier, MAP is where the maximum of the oscillometric envelope occurs; diastolic is where the oscillation amplitude is at 60% of the MAP oscillation size on the lower pressure side of the oscillometric envelope. Once again a curve fit could be done, but, in this embodiment, only to estimate diastolic and MAP are needed. Interpolation between steps can be used by means of the fitted curve to improve the diastolic estimate since the measured envelope data does not typically provide a point that is exactly 60% of the maximum oscillation amplitude while also being at a pressure step.

In an example the pressure cuff is inflated at a rate so that a sufficient number of oscillometric pulses can be found. Note that by calculating the initial inflation pressure an overall faster determination will result even though in some cases the inflation period may take a little longer to get enough detailed oscillometric information during inflation to do a good curve fit. An advantageous use of the invention is in the case of a large cuff applied to a patient who is hypertensive. If the volume of the cuff is large, pumping to the initial inflation pressure will naturally take longer allowing acquisition of the needed oscllometric pulses. Generally, there is no need to develop a special cuff inflation strategy to control the pump in some dynamic or elaborate way. If the inflation does occur so fast that the default initial inflation pressure is attained before a new and better initial inflation pressure is estimated, then the NIB system can proceed as usual with the determination. If a better initial inflation pressure is found before the cuff reaches the default initial inflation pressure then the NIB system can use the newly calculated initial inflation pressure. This provides an opportunity to accelerate the determination, but only if the situation allows it.

FIG. 8 is a flowchart illustrating the course of actions used in estimating the initial inflation pressure as in an embodiment of the invention. At step 810, the determination of blood pressure using an NIB system is started. At step 820, the NIB system is set with an initial inflation pressure from an earlier blood pressure estimation or with a default initial inflation pressure. At step 830, the cuff inflation towards the set initial inflation pressure is started. At step 840, the microprocessor checks whether the cuff pressure has reached the set initial inflation pressure. If the cuff pressure reaches the set initial inflation pressure before calculating a new initial inflation pressure, the process of trying to calculate a new initial inflation pressure is terminated and the device proceeds with deflation of the cuff as indicated in step 890. At step 850, if the cuff pressure has not yet reached the set initial inflation pressure, then the microprocessor checks whether a sufficient number of oscillations have been received to sufficiently define an oscillometirc envelope for estimating the systolic pressure. If a sufficient number of oscillations, preferably and minimally three, have not been received for estimating the quick systolic, the microprocessor will check whether the set initial inflation pressure has been reached and if not, it will continue monitoring for oscillations. If the cuff pressure has already reached the set initial inflation pressure, then the device will proceed with deflation. At step 860, the microprocessor checks whether a pre-defined function can be used for curve fitting oscillation data that has been obtained so far to that point in the inflation process. If the curve fitting is able to be done before the cuff pressure reaches the set initial inflation pressure, then at step 870 a new initial inflation pressure is calculated. If the curve fitting is not able to be done, the algorithm will return to step 840 to see if cuff inflation should cease. If enough information is available so that a new initial cuff pressure is able to be calculated, then the algorithm enters step 870. At step 880, the cuff is inflated to the new initial inflation pressure. At step 890, the cuff pressure is deflated from the initial inflation pressure in the normal fashion to get blood pressure estimates for publication to the user.

Thus various embodiments of method of estimating an initial inflation pressure and controlling the inflation of the cuff are provided. While the invention has been described with reference to preferred embodiments, those skilled in the art will appreciate that certain substitutions, alterations and omissions may be made to the embodiments without departing from the spirit of the invention. Accordingly, the foregoing description is meant to be exemplary only, and should not limit the scope of the invention as set forth in the following claims.

We claim:

1. A method of calculating an initial inflation pressure, while monitoring blood pressure, comprising the steps of:
(a) inflating a blood pressure cuff;
(b) monitoring for the presence of oscillometric pulses during the inflation of the blood pressure cuff;
(c) estimating a quick systolic blood pressure for a patient based on the oscillometric pulses detected during the inflation of the blood pressure cuff; and
(d) defining the initial inflation pressure as a predetermined pressure above the estimated systolic pressure;
wherein the systolic blood pressure is estimated by curve fitting with a pre-defined function having a physiologically-expected shape of an oscillometric envelope, the curve fitting being done by using a plurality of oscillometric pulses along with corresponding cuff pressures obtained during an early part of inflation.

2. A method as in claim 1, wherein the step of inflating the blood pressure cuff comprises increasing cuff pressure at a rate such that the initial inflation pressure, is defined before the cuff pressure reaches a default initial inflation pressure.

3. A method as in claim 1, wherein the step of monitoring for the presence of oscillometric pulses, comprising the steps of:
(a) receiving a plurality of oscillations from a pressure transducer during the inflation of the blood pressure cuff; and
(b) filtering a cuff pressure waveform to extract the oscillometric pulses.

4. A method as in claim 3, wherein while monitoring for the presence of oscillometric pulses, further comprises defining an oscillometric envelope using amplitudes of the oscillometric pulses along with corresponding cuff pressures.

5. A method as in claim 1, wherein estimating the systolic blood pressure, comprises the steps of:
(a) finding the function parameters within the pre-defined function with curve fitting using oscillometric pulses along with corresponding cuff pressures obtained during the inflation of the cuff;
(b) obtaining a mean arterial pressure and a diastolic pressure from the fitted curve; and
(c) estimating the systolic pressure from the estimated mean arterial pressure and a diastolic pressure.

6. A method as in claim 5, wherein estimating systolic blood pressure from the estimated mean arterial pressure and diastolic pressure, further comprises the steps of:
(a) selecting at least one oscillometric pulse from the plurality of oscillometric pulses obtained during the inflation;
(b) calibrating the selected oscillometric pulse with the obtained mean arterial pressure and diastolic pressure; and
(c) estimating the systolic pressure from the calibrated oscillometric pulse.

7. A method as in claim 5, wherein estimating systolic blood pressure from the estimated mean arterial pressure and diastolic pressure, further comprises estimating systolic pressure using any mathematical formula relation of mean arterial pressure and diastolic pressure with systolic pressure.

8. A method as in claim 5, wherein the quick systolic pressure is estimated based on data gathered from only part of the inflation pressure range.

9. A method as in claim 1, wherein defining the initial inflation pressure comprises the step of calculating the initial inflation-pressure before the cuff pressure achieves the default initial inflation pressure.

10. A method as in claim 9, which further comprises the step of terminating the process for attempting the calculation of the initial inflation pressure, if the cuff pressure reaches the default initial inflation pressure before calculating the new initial inflation pressure.

11. A method of monitoring blood pressure for a patient, comprising the steps of:
(a) providing a non-invasive blood pressure (NIB) monitor having a selectively inflatable and deflatable blood pressure cuff and at least one pressure transducer for detecting oscillometric pulses;
(b) inflating the blood pressure cuff;
(c) generating an oscillometric envelope from the oscillometric pulses obtained when varying cuff pressure during the inflation of the blood pressure cuff;
(d) estimating the MAP and diastolic pressures for the patient by curve fitting using a pre-defined function having a physiologically-expected shape of an oscillometric envelope and using a plurality of oscillometric pulse amplitudes obtained during inflation along with their corresponding cuff pressures so that the values of parameters within the function are found to completely specify that same function; and
(e) terminating the inflation of the blood pressure cuff at an initial inflation pressure above the estimated systolic blood pressure, where the systolic pressure is estimated from a calibrated cuff pressure oscillation or from a mathematical formula that relates systolic to diastolic and MAP.

12. A method as in claim 12, wherein the oscillometric envelope used for curve fitting is obtained from a portion of the inflation period.

13. A method as in claim 12, wherein the systolic pressure is estimated during the inflation of the cuff, at a point in time when the amplitude of the obtained oscillometric pulses start decreasing.

14. A method as in claim 11, further comprising:
(a) deflating the blood pressure cuff from the initial inflation pressure;
(b) monitoring for oscillometric pulses from the pressure transducer during the deflation of the blood pressure cuff from the initial inflation pressure; and
(c) determining the systolic pressure, mean arterial pressure and diastolic pressure of the patient based upon the oscillometric pulses detected during the deflation of the blood pressure cuff from the initial inflation pressure.

15. A method of controlling inflation of a cuff during monitoring blood pressure of a patient using a NIB system comprising the steps of:
(a) providing the patient with a selectively inflatable and deflatable non-invasive blood pressure cuff arranged to be worn about a limb of the patient and operatively connected to a non-invasive blood pressure monitor;
(b) estimating a systolic blood pressure for the patient from oscillometric information measured from only part of the inflation pressure range and based on using a pre-defined function having a physiologically-expected shape of an oscillometric envelope for curve fitting to a plurality of oscillometric pulse amplitudes along with their corresponding cuff pressures; and
(c) controlling inflation of the blood pressure cuff based on the calculated initial inflation pressure, the initial inflation pressure being obtained from an estimated systolic pressure, during inflation of the blood pressure cuff.

16. A method as in claim 15, wherein the systolic pressure is estimated from a calibrated cuff pressure oscillation or from a mathematical formula that relates systolic to diastolic and MAP.

17. A method as in claim 15, wherein controlling inflation further comprises the steps
(a) setting an initial inflation pressure at the beginning of inflation of the cuff
(b) monitoring the status of cuff pressure during inflation, the status being assigned based on whether the cuff pressure reached the set initial inflation pressure;
(c) terminating the inflation of the blood pressure cuff at a calculated initial inflation pressure, if the new initial inflation pressure is calculated before the cuff pressure achieves the first set initial inflation pressure; and
(d) deflating the blood pressure cuff from the initial inflation pressure.
